(19) United States
(12) Patent Application Publication
(10) Pub. No.: US 2004/0138646 A1
(43) Pub. Date: Jul. 15, 2004

(54) Device for administering a substance transdermally

(76) Inventor: Thomas Walla, Wien (AT)

Correspondence Address: GREENBLUM & BERNSTEIN, P.L.C., 1950 ROLAND CLARKE PLACE, RESTON, VA, 20191, US

(21) Appl. No.: 10/477,754

(22) PCT Filed: May 23, 2002
(86) PCT No.: PCT/AT02/00158

(30) Foreign Application Priority Data
May 25, 2001 (AT) A 833/01

(51) Int. Cl. A61N 1/30; A61K 9/22

(52) U.S. Cl. 604/890.1; 604/20

(57) ABSTRACT

The invention relates to a device for transdermal administration of substances, such as medicinal products in the form of a liquid, ointment or gel, in particular by means of iontophoresis. The substance is absorbed through an electric field that can be produced with a defined direct current between two electrodes (2). It consists of capsule-like, hermetically sealed container (1) that can be inserted into orifices of the body, with at least two electrodes (2) for producing the electric d.c. field on its outside. Above the electrodes (2) a device (3) is provided to contain the substance to be administered. The container (1) is in contact with mucous membrane and/or skin in an orifice of the body, in particular in the urogenital and/or vaginal and/or anal tract and/or in the mouth and/or ears and/or nose.

The invention relates to a device for the transdermal administration of substances such as medicinal products in the form of a liquid, ointment or gel, in particular by means of iontophoresis, whereby the substance is absorbed through an electric field that can be produced by a defined direct current between two electrodes.

The Natural Healing Dictionary, 2000 MZ-Verlag, defines iontophoresis as the application of direct current to enhance the absorption of medicinal products applied to the skin. Ionisation of the medicinal products must be possible. The electrode is applied directly to the skin to which the medicinal product has been applied. Depending on the specifications, either the anode or the cathode is in contact with the skin. The quantity of the medicinal product absorbed depends on the intensity of current, the duration of treatment and the electrode surface size. This method exploits the electrical charge of ions or molecules. Since opposite charges attract each other, negative charges migrate to the positive electrode and positive charges migrate to the negative electrode when direct current is applied.

Iontophoresis is used for example in muscular or skeletal disorders by administering ointments to the body.

Iontophoresis with tap water has also been proved as a method of treatment for excessive sweating of the hands, feet and armpits.

However, other conditions are also known, especially among women, that are alleviated or healed primarily by means of surgical intervention. Incontinence, for example, is a widespread condition that reduces the quality of life drastically. The cause of these symptoms is to be found in a weakening of the pelvic floor muscles, resulting among other things in the angle between urinary bladder and ureter changing and restricted functioning of the sphincter, with the urethra sagging due to the weakened pelvic floor. Weakening of the pelvic floor muscles can be caused either by childbirth or due to disposition or age.

Efforts are now being made to cure this condition with conservative, non-surgical methods of treatment. Within the scope of these non-surgical methods of treatment, mechanical training of the pelvic floor muscles, electrical stimulation and drug therapies will be used.

The aim of the invention is to provide a device as described above that allows suitable medicinal products for the alleviation or healing of disorders to be administered specifically in the causal area of the disorders in the form of non-surgical treatment.

The problem is solved by this invention.

The device according to the invention is characterised by the fact that a capsule-like, hermetically sealed container that can be introduced into orifices in the body is provided, with at least two electrodes for the production of a d.c. field on the outside, and that a device to contain the substance to be administered is provided above the electrodes. With this invention it is for the first time possible, depending on the cause of the disorders and the location, to administer medicinal products, in particular iontophoresis preparations, specifically through orifices in the body by means of iontophoresis. With the interaction between iontophoresis device and iontophoresis preparation, optimal specific treatment can be provided.

This is supported by the fact that the arrangement of the electrodes is based on an appropriate mathematical simulation of conditions in the selected orifice. An even circulation of the area to be treated and thus the optimised administration of the preparations, even in the depths of the orifices, is thus guaranteed.

A further advantage of the invention can be seen in the fact that the treatment can be carried out without interrupting daily routine, since the device according to the invention is left in the orifice and no external equipment is required during the treatment.

According to a special feature of the invention, the container is in contact with mucous membrane and/or skin in an orifice of the body, in particular in the urogenital and/or vaginal and/or anal tract and/or in the mouth and/or ears and/or nose. Due to the effect of the electric field and the sensitivity of the skin or mucous membrane, a promising treatment is possible that could avoid the need for surgery.

In accordance with one embodiment of the invention a programmable source of power is connected to the electrodes. This allows the selection of iontophoresis parameters such as intensity of current, pulse ratio and duration of application in accordance with the treatment plan.

In accordance with a further embodiment of the invention, the surface of the capsule-like container can be heated to a defined temperature through an integrated, electrical heating unit. By heating the surface to a defined temperature, the effect of the medicinal product can be enhanced.

In accordance with a special embodiment of the invention, power is supplied by a rechargeable battery that is integrated in the hermetically sealed container. The advantage of this embodiment speaks for itself. During the treatment pauses, i.e. when the device is not inserted in an orifice, the rechargeable batter is supplied with energy from an external charging device, whereby safe handling of the device according to the invention is guaranteed.

In accordance with a further embodiment, the power is supplied inductively by an external system. The advantage of this embodiment is that the device according to the invention does not need to be removed from the orifice. Based on the current state of the art, such an energy supply is perfectly conceivable.

In accordance with a special embodiment of the invention, the parameters of the programmable power source and temperature of the integrated heating unit can be set via an interface with or without contacts. Thus it is possible to ensure that only authorised persons, such as e.g. the physician, can adjust or change the parameters to the patient's individual needs or the treatment plan.

In accordance with a further embodiment of the invention, the reference value and form of current can be preset in digital form in accordance with the settings via an interface with or without contacts. This allows for very simple programming.

In accordance with a further embodiment of the invention, the power source compares the set reference value with the measured actual value and regulates the difference. Thus the treatment parameters can be assured in a very simple manner.

In accordance with a special embodiment of the invention it is possible with appropriate safety measures at the power source output to avoid the no-load voltage from exceeding a set limit, e.g. 80 V. This guarantees safe operation and also compliance with a wide range of standards.

In accordance with an advantageous embodiment of the invention, the duration of current can be programmed digitally via an interface with or without contacts. This allows treatment parameters to be set in a very easy manner.

In accordance with a further embodiment of the invention, the current increases gradually at the beginning of the programmed time, until the programmed reference value is reached, and then gradually decreases to zero at the end. Thus irritation of the skin or mucous membrane can be avoided.

In accordance with a further advantageous embodiment of the invention, the time remaining of the programmed total time can be stored in the case of an external interruption, and the remaining time is available for the release of energy when the power is switched on again. Thus the treatment time can be maintained in an optimal and comfortable manner.

In accordance with a special embodiment of the invention, the power source can be switched on and off with a contact switch. Such switches are easy to use and they are extremely suitable for the overall design of the device according to the invention, namely a capsule-like, hermetically sealed container.

In accordance with one embodiment of the invention, a temperature and time control programmable via an interface with or without contacts can be realised to control the electric heating unit integrated in the wall of the capsule-like container. This also allows various treatment parameters to be changed in a very easy way in order to achieve a good treatment result.

In accordance with a special feature of the invention, the device for containing the substance to be administered, in particular a medicinal product, is made for example of open-pored foam strips that are soaked in and/or covered with the defined substance to be administered. This design contributes towards optimal administration of the medicinal product. Moreover the use of such foam strips has an extremely high economic aspect.

In accordance with another embodiment of the invention, gel-like shaped parts are provided as substance carriers. Such shaped parts can also be used in a very advantageous manner.

In accordance with another embodiment of the invention, nonwoven fabrics or the like are provided as substance carriers. Such substance carriers are also suitable for use.

In accordance with a special feature of the invention, at least one pressure sensor to measure tissue tone is arranged on the outside of the hermetically sealed container. With such a sensor, the success of treatment can be measured indirectly. The effect of the medicinal product is monitored by measuring tissue tone, so to speak.

In accordance with a further embodiment of the invention, the measurements by the pressure sensor can be recorded in a permanent memory. Thus the effect of the medicinal product can be observed over a given period of time.

In accordance with a further embodiment of the invention, the recorded measurements can be read out via an interface with or without contacts. Thus the success of treatment can be made reproducible and transparent.

In the following, the invention is explained in more detail based on the embodiment illustrated in the figure.

The figure shows:

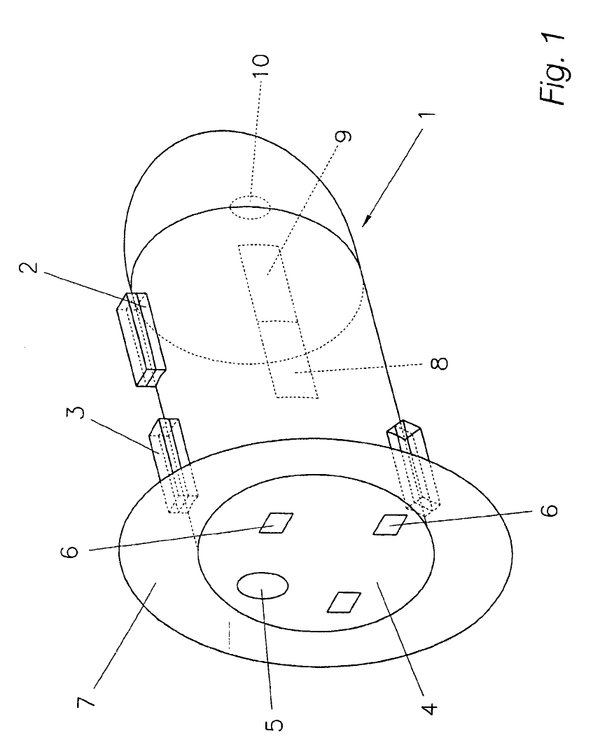

FIG. 1: a schematic illustration of the device,

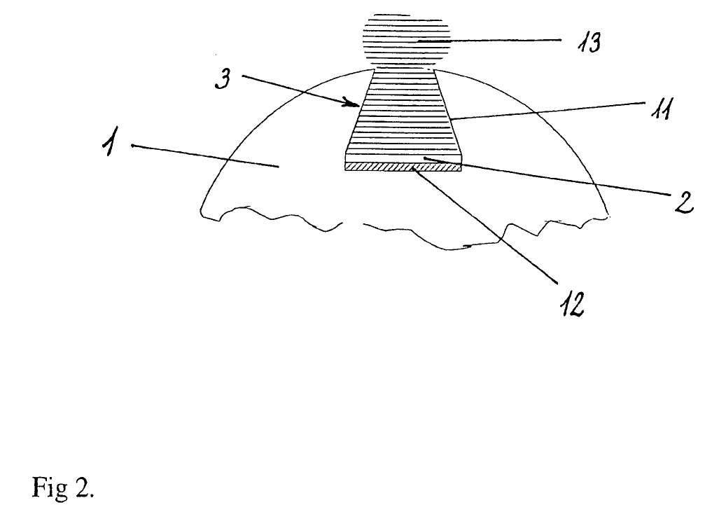

FIG. 2: a detailed view of one electrode, and

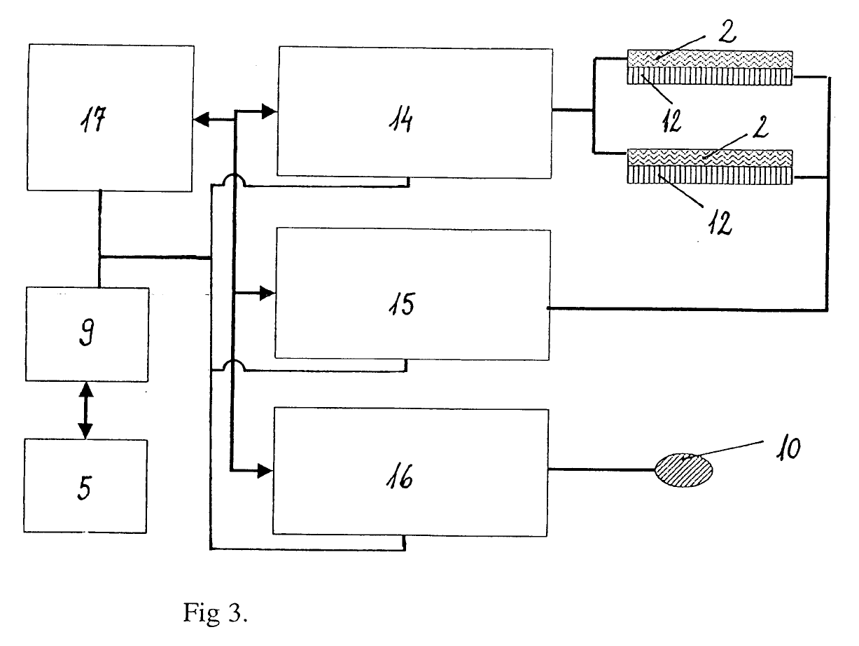

FIG. 3: a schematic circuit diagram of the device.

By way of introduction, it is noted that the same parts are allocated the same reference numbers and the same component names, whereby the disclosures contained throughout the description can be applied by analogy to the same parts with the same reference numbers or same component names. Furthermore, position details given in the description, e.g. top, bottom, side, etc., relate to the figure being described and illustrated at the time and with a change of position should be transferred accordingly to the new position. Moreover, individual features or combinations of features from the embodiment illustrated and described can represent independent inventive solutions or solutions according to the invention in themselves.

The problem forming the basis of the separate solutions according to the invention can be taken from the description.

FIG. 1 shows a device that can be used as a vaginal iontophoresis device. This vaginal iontophoresis device is introduced into the vaginal tract and serves to administer substances, in particular medicinal products, through the mucous membrane. The vaginal iontophoresis device consists of a cylindrical, capsule-like, hermetically sealed container 1, which has at least two electrodes 2 on the outside. Above the electrodes 2 a device 3 is provided to contain the medicinal product to be administered. The cylindrical container 1 is sealed at one end with a capsule-like hemisphere. The other end of the container 1 is hermetically sealed with a cover plate 4, whereby one contact switch 5 and interface contacts 6, for example for the transmission of data, are integrated in this cover plate 4. In this case the cover plate 4 is fitted with a twist protection 7, which can also be used as an insertion aid.

On the inside of the container 1 of the vaginal iontophoresis device there is an electronic system 8, which is supplied for example by a rechargeable integrated battery 9, whereby presettable constant currents for example in the range from 0.1 mA to 10 mA direct current can be produced. Depending on the medicinal product used, the pulse ratio can be preset for example within a range from 1 to 15 up to constant current. Naturally the power can also be supplied inductively by an external system.

The arrangement of the electrodes 2 is, based upon a mathematical simulation of conditions in the female urogenital tract, such that an even circulation of blood in the pelvic floor region and thus optimised administration of the products is guaranteed even in the depths of the pelvic floor muscles.

In the container 3, the medicinal product or preparation to be administered is brought into physical contact with the electrodes 2 in the form of medicinal strips, i.e. open-pored foam strips soaked with the relevant concentration of the preparation. Thus it is easily possible, by exchanging the single-use medicinal strips, to apply various concentrations and types of preparations within the scope of a therapy plan. The medicinal strips are extremely easy to insert into the container 3 and can be inserted by the patient.

Gel-like shaped parts, nonwoven fabrics or the like can also be used as substance carriers.

On the outside of the hermetically sealed container 1 there is at least one pressure sensor 10 to measure the tissue tone. With such a pressure sensor 10, the success of treatment can be measured indirectly. The effect of the medicinal product is monitored by measuring tissue tone, so to speak.

FIG. 2 shows the area of the electrode 2 and the device 3 above it for the medicinal strips in the casing of container 1 in detail. The casing of container 1 has a groove 11, for example a dovetail groove, at the bottom of which a heating unit 12 is arranged. Electrode 2 is provided above this heating unit 12. A substance carrier 13, in particular a medicinal strip, is placed above the electrode 2.

The electronic system 8 of the unit described in FIG. 1 is explained in more detail using the schematic circuit diagram shown in FIG. 3. The electronic system 8 comprises the energy supply, preferably a rechargeable battery 9 that is switched on and off using the contact switch 5. Moreover, a programmable power source 14 that is connected to the electrodes 2, a programmable temperature control 15 that is connected to the heating unit 12, a memory 16 for the values measured by the pressure sensor 10, and an interface 17 with the interface contacts 6 are supplied with energy from the energy supply.

The electrodes 2 are controlled via the programmable power source, and the heating units 12 are controlled via the programmable temperature control 15. Thus the reference value and form of current can be predefined in digital form in accordance with the settings via the interface 17. Naturally it is also possible for the power source 14 to compare the set reference value with the measured actual value and to regulate the difference. It is also conceivable and of advantage for the duration of the current to be programmable in digital form via the interface 17. Thereby the current can increase gradually at the beginning of the programmed time until the programmed reference value is reached, and then decrease gradually to zero at the end.

In the event of an external interruption of the set time, the time remaining of the programmed total time can be saved and is available for power release when the power supply is switched on again. By means of appropriate protection measures at the output of the power source 14, it is possible to prevent the no-load voltage from exceeding a certain limit, for example 80 V.

The programmable power source 14 and the programmable temperature control 15 can thus be set via the interface 17, which may be with or without contacts, and its interface contacts 6. Naturally a programmable temperature control for temperature and time to control the electric heating unit 12 integrated in the wall of the capsule-like container 1 can be realised via the interface 17.

The measurements by the pressure sensor 10 can be recorded in a permanent memory 16, whereby the recorded measurements can be read out via the interface 17.

The vaginal iontophoresis device can be supplemented on the one hand by a charging device and on the other hand by a setting or testing device. The charging device supplies the inserted vaginal iontophoresis device with energy and serves to recharge the integrated battery 9. The charging time depends on current, pulse ratio and duration of application. This is displayed optically when the cleaned vaginal iontophoresis device is placed in the charging device. The setting and testing device, which is available only to physicians, allows the physician on the one hand to set the current and pulse ratio—depending on the medicinal products to be administered—and the patient's individual needs. On the other hand this device allows the proper functioning of the vaginal iontophoresis device and the programmed settings to be tested. The patient can only switch the vaginal iontophoresis device on and off, clean it, insert and remove the prescribed medicinal strips, and recharge the battery 9 using the charging device. The patient cannot change the iontophoresis parameters programmed by the physician. For this treatment, in particular esterified steroids and estriol salts, as well as special preparations to stimulate circulation in the pelvic floor are used. The advantages of this new device can be seen mainly in the fact that treatment is possible without interruptions to daily routine, since the vaginal iontophoresis device is used intravaginally and does not require any external equipment during its operation. Depending on the incontinence symptoms and patient's age, various combinations of active substances can be used, whereby the medicinal strips are ready for single use and easy for the patient to insert. Based on the variable pulse ratio in combination with the finely adjustable current, an optimal deep effect for transfusion of the pelvic floor muscles can be achieved with each preparation and for the various degrees of incontinence symptoms.

Last but not least it is expressly noted that the container 1 is in contact with mucous membrane and/or skin in an orifice of the body, in particular in the urogenital and/or vaginal and/or anal tract and/or in the mouth and/or ears and/or nose.

For form's sake, it is noted that for a better understanding of the structure of the device, the parts and their components are illustrated partly untrue to scale and/or are enlarged and/or made smaller.

1. Device for the transdermal administration of substances, such as medicinal products in the form of a liquid, ointment or gel, in particular by means of iontophoresis, whereby the substance is absorbed through an electric field that is produced by a defined direct current between two electrodes, and where a capsule-like, hermetically sealed container that can be introduced into orifices of the body is provided, which has a container for the substance to be administered and/or a substance carrier soaked in or covered with the substance to be administered, and where this container is inserted into an orifice of the body and is in contact with the mucous membrane or skin of this orifice, especially in the urogenital and/or vaginal and/or anal tract and/or the mouth and/or ears and/or nose, characterised by the fact that the container (1) has at least two electrodes (2) to produce the electric field on the outside, and that a programmable power source (14) is provided in the container (1), which is connected to the electrodes (2).

2. Device in accordance with claims 1, characterised by the fact that the surface of the capsule-like container (1) can be heated to a defined temperature by means of an integrated, electrical heating unit (12).

3. Device in accordance with claim 1 or 2, characterised by the fact that the power supply is provided in the form of a rechargeable battery (9) integrated in the hermetically sealed container (1).

4. Device in accordance with claim 1 or 2, characterised by the fact that the energy supply is provided inductively by an external system.

5. Device in accordance with at least one of claims 1 to 4, characterised by the fact that the parameters of the programmable power source (14) and temperature of the integrated heating unit (12) can be set via an interface (17) with or without contacts.

6. Device in accordance with at least one of claims 1 to 5, characterised by the fact that the reference value and form of current can be defined digitally in accordance with the settings via an interface (17) with or without contacts.

7. Device in accordance with at least one of claims 1 to 6, characterised by the fact that the power source (14) compares the set reference value with the measured actual value and regulates the difference.

8. Device in accordance with at least one of claims 1 to 7, characterised by the fact that with appropriate protection measures at the output of the power source (14) it is possible to prevent the no-load voltage from exceeding a certain limit, for example 80 V.

9. Device in accordance with at least one of claims 1 to 8, characterised by the fact that the duration of the current can be programmed digitally via an interface (17) with or without contacts.

10. Device in accordance with at least one of claims 1 to 9, characterised by the fact that at the beginning of the programmed time the current increases gradually until the programmed reference value is reached, and then decreases gradually to zero at the end.

11. Device in accordance with at least one of claims 1 to 10, characterised by the fact that the time remaining of the programmed total time can be stored in the case of an external interruption, and the remaining time is available for the release of energy when the power is switched on again.

12. Device in accordance with at least one of claims 1 to 11, characterised by the fact that the power source (14) can be switched on and off with a contact switch (5).

13. Device in accordance with at least one of claims 5 to 12, characterised by the fact that a temperature and time control programmable via an interface (17) with or without contacts can be realised to control the electric heating unit (12) integrated in the wall of the capsule-like container (1).

14. Device in accordance with at least one of claims 1 to 13, characterised by the fact that the container (3) for the substance to be administered, in particular a medicinal product, is made for example of open-pored foam strips that are soaked in and/or covered with the defined substance to be administered.

15. Device in accordance with at least one of claims 1 to 14, characterised by the fact that gel-like shaped parts are provided as substance carriers (13).

16. Device in accordance with at least one of claims 1 to 13, characterised by the fact that nonwoven fabrics or the like are provided as substance carriers (13).

17. Device in accordance with at least one of claims 1 to 16, characterised by the fact that there is at least one pressure sensor (10) to measure tissue tone on the outside of the hermetically sealed container (1).

18. Device in accordance with claim 17, characterised by the fact that the measurements by the pressure sensor (10) can be recorded in a permanent memory (16).

19. Device in accordance with claim 18, characterised by the fact that the recorded pressure measurements can be read out via an interface (17) with or without contacts.
